(19) United States
(12) Patent Application Publication
(10) Pub. No.: US 2026/0026831 A1
(43) Pub. Date: Jan. 29, 2026

(54) SURGICAL INSTRUMENT

(71) Applicant: Richard Wolf GmbH, Knittlingen (DE)

(72) Inventors: Eberhard KÖRNER, Knittlingen (DE); Silas MAUL, Pforzheim (DE); Vincent FRATTINI, Karlsruhe (DE)

(21) Appl. No.: 18/994,704

(22) PCT Filed: Jul. 11, 2023
(86) PCT No.: PCT/DE2023/200141

§ 371 Date: Jan. 15, 2025

(30) Foreign Application Priority Data
Jul. 18, 2022 (DE) 10 2022 207 325.8

(51) Int. Cl. A61B 17/29; A61B 17/3201

(52) U.S. Cl. CPC A61B 17/2909; A61B 17/3201; A61B 2017/2912; A61B 2017/2929; A61B 2017/2944

(57) ABSTRACT

A surgical instrument is for gripping tissue within a body, and includes an actuation device with one grip part movably mounted relative to another in an opening and closure direction and a tool head with two tool parts. One tool part is movable relative to the other along an opening and closure direction. A shank extends between the actuation device and the tool head, with proximal end connected or connectable to the actuation device and distal end connected or connectable to the tool head. The shank includes a transmission axially movable in the opening and closure direction to transmit force from the first grip to the first tool. An overload protection device includes a spring element limiting force transmitted onto the first tool part in the closure direction, and a toggle lever mechanism via which the spring element biases the transmission element in the closure direction.

### CROSS REFERENCE TO RELATED APPLICATIONS

This application is a United States National Phase Application of International Application PCT/DE2023/200141, filed Jul. 11, 2023, and claims the benefit of priority under 35 U.S.C. § 119 of German Application DE 10 2022 207 325.8, filed Jul. 18, 2022, the entire contents of which are incorporated herein by reference.

### TECHNICAL FIELD

The present invention pertains to to surgical instruments for the surgical gripping, pinching and/or cutting of tissue within an organic body, in particular laparoscopic instruments.

Surgical instruments for endoscopic operations, such as laparoscopic instruments are often configured as thinly as possible, in order to be able to carry out the surgical operation as minimal-invasively as possible for the patient. The tool head of such a surgical instrument therefore typically comprises relatively delicate tool parts such as jaw parts of a (pinching) forceps or blades of a pair of scissors. For this reason, there basically exists the risk of the manual force of the user which is transmitted onto the tool head being so large that delicate tool parts break.

### BACKGROUND

It is therefore known for such manually operable surgical instrument to comprise a force limitation device or overload protection device which significantly reduces the risk of a breakage of the tool parts.

For example, such overload protection devices are known from DE 101 10 106 B4, DE 103 28 514 B3, DE 299 17 554 U1, U.S. Pat. No. 5,009,661 A, DE 44 11 099 C2 or DE 297 13 490 U1.

The known overload protection devices however have the disadvantage that the manual force which is transmitted onto the tool head is relatively small given small opening angles of the tool. For this reason, small pieces of tissue cannot be gripped, pinched off and/or cut up with the necessary force in certain situations. It is particularly in the case of an often necessary tweezer grip with the outermost distal end of the tool head that the gripping, pinching or cutting force is particularly low.

The reason for this lies in the fact that the spring characteristic of a spring which usually serves as an overload protection device is a line through the origin and the spring only has a small spring force at the beginning of the spring deflection. If, given small opening angles of the tool, the further range of motion of the actuation lever is not sufficient in order to increase the spring force then the manual force which is transmitted onto the tool head is relatively small. A small piece of tissue could therefore escape from the tweezer grip or not be successfully pinched off or cut up.

### SUMMARY

It is therefore the object of the present invention to protect the tool head of the surgical instrument on the one hand from overload and on the other hand to provide a sufficiently high force transmission given smaller opening angles of the tool.

This object is achieved with features according to the invention. Preferred embodiments can be derived from the description, figures and claims.

According to the present disclosure, a surgical instrument is provided for the surgical gripping, pinching and/or cutting of tissue within an organic body, wherein the surgical instrument comprises:
an actuation device with two grip parts, wherein at least a first of the two grip parts is movably mounted relative to a second of the two grip parts in a translatory and/or rotatory manner in an opening and closure direction,
a tool head with two tool parts, wherein at least a first of the two tool parts is movably mounted relative to a second of the two tool parts in a translatory and/or rotatory manner in an opening and closure direction,
a shank which extends along a longitudinal axis between the actuation device and the tool head, wherein a proximal end of the shank is connected or connectable to the actuation device, and wherein a distal end of the shank is connected or connectable to the tool head, wherein the shank comprises a transmission element which is axially movable in an opening and closure direction for transmitting a manual force from the first grip part onto the first tool part, and
an overload protection device with a spring element for limiting the manual force which is transmitted onto the first tool part in the closure direction.

The surgical instrument is characterised in that the overload protection device comprises a toggle lever mechanism, via which the spring element biases the transmission element in the closure direction.

On account of the biasing, it is ensured that immediately at the beginning of the deflection of the spring element an adequately high force transmission onto the tool is achieved independently of the opening angle of the tool head. This means that given small opening angles of the tool head, at least the biasing force of the spring element is transmitted onto the tool. By way of a suitable design of the parameters of the toggle lever mechanism and of the spring element, a desired biasing force can be ensured, so that even the smallest of pieces of tissue can be successfully pinched and/or cut up in the tweezer grip, which is to say cannot slip away out of the tweezer grip.

A further advantage of the toggle lever mechanism is the fact that the force transmission onto the tool on the spring path can even be reduced given larger pieces of tissue which require a larger opening angle of the tool head. For larger piece of tissue, the tweezer grip with the distal end of the tool head indeed is not necessary at all and in the used middle or proximal region of the tool parts in principle less force is required due to the lever effect. Furthermore, in practise large, hard pieces of tissue hardly play any role at all.

Optionally, the toggle lever mechanism can be articulately connected to the transmission element in a toggle point. This is advantageous in order, given an uncomplicated as possible construction of the toggle lever mechanism and a minimal variety of parts, to introduce the force from the spring element onto the transmission element in the direction of the longitudinal axis.

Optionally, the toggle lever mechanism can comprise two distal bearing points which are radially distanced to the longitudinal axis and via which the toggle lever mechanism is articulately connected in each case to one of two spring bows of the spring element. Herewith, a construction of the overload protection device which is essentially symmetrical to the longitudinal axis can be achieved, wherein the spring force is distributed uniformly onto the two spring bows. The spring bows can be part of a single-piece spring element, wherein the spring bows resiliently pivot radially outwards amid the bending of the spring element. The spring element can therefore form two resilient proximal solid-body joints, from which the spring bows each extend to the respective distal bearing point.

Optionally, the toggle point can be arranged proximally of the distal bearing points. By way of this, the toggle lever mechanism in the toggle point defines a distally opened opening angle. If the distal bearing points are pulled proximally in the closure direction and the toggle point feels a resistance to a further movement in the closure direction, said resistance being caused by a piece of tissue in the tool, then the opening angle of the toggle lever mechanism enlarges against the biased spring force of the spring element.

Optionally, the spring element can comprise a proximal bearing point, via which the spring element is articulately connected to the first grip part. This proximal bearing point preferably lies on the longitudinal axis of the shank and moves proximally in the closure direction and distally in the opening direction. The first grip part preferably pulls the spring element proximally at the proximal bearing point when the first grip part is moved in the closure direction.

Optionally, the proximal bearing point can be arranged proximally of the toggle point. Herewith, the spring element at the toggle point pulls the transmission element proximally in the closure direction via the toggle lever mechanism. Given a proximal movement of the transmission element, the tool parts of the tool head then close.

Optionally, the spring element can be coupled to the first grip part in a manner such given a movement of the first grip part in the closure direction its spreads radially outwards counter to the intrinsic spring tension amid the opening of the toggle lever mechanism, as soon as the further movement of the first tool part is blocked in the closure direction by tissue. Preferably, the friction resistance for moving the transmission element and the tool parts is so low that no spreading of the spring element results without a blocking of the tool parts by tissue.

Optionally, the spring element can comprise two spring bows which on opposite sides extend laterally of the longitudinal axis from the first grip part to the toggle lever mechanism. Herewith, a configuration of the overload protection device which is essentially symmetrical to the longitudinal axis can be achieved, wherein the spring force is uniformly distributed onto the two spring bows.

Optionally, the spring element can comprise a guide opening which extends along the longitudinal axis and in which the toggle point is mounted in an axially movably guided manner. The opening angle region of the toggle lever mechanism and herewith the deflection region of the spring element can be defined by the length and position of the guide opening. If for example the toggle point is located at the proximal end of the guide opening, then the opening angle of the toggle lever mechanism and thus the spring deflection is minimal. If the toggle point is located for example at the distal end of the guide opening, then the opening angle of the toggle lever mechanism and herewith the spring deflection is maximal. The guide opening is preferably an elongate hole in the spring element, said elongate hole extending along the longitudinal axis. An over-tensioning of the spring element into a non-elastic region is herewith ruled out since the toggle point abuts on the distal end of the guide opening. The spring element can be biased by way of a suitable position of the proximal end of the guide opening and the length of the toggle lever legs of the toggle lever mechanism. This means that the overcoming of the biasing force is necessary in order to move the toggle point distally away from the proximal end of the guide opening.

Optionally, the spring element is biased with a biasing force when the toggle lever mechanism is at a minimal opening angle, wherein the opening angle of the toggle lever mechanism widens counter to the biasing force as soon as the further movement of the first tool part is blocked in the closure direction by way of tissue. Herein, the manual force which is transmitted onto the first tool part in the closure direction can preferably be greater than the biasing force in a first opening angle region of the toggle lever mechanism and can be lower than the biasing force in a second opening angle region of the toggle lever mechanism which lies above the first opening angle region. Given large pieces of tissue, specifically no tweezer grip and thus less force is necessary for the tool. Only a force which changes only to a limited extent over the complete opening angle region of the toggle lever mechanism or over the complete spring path or over the complete path of the toggle point in the guide opening can be transmitted onto the transmission element.

Optionally, the toggle lever mechanism can comprise two equally long toggle lever legs which are articulately connected to one another in the toggle point, wherein the length of the toggle lever legs is larger than an axial range of motion of the toggle point relative to a proximal bearing point of the spring element, via which bearing point the spring element is articulately connected to the first grip element. Alternatively to this, the toggle lever mechanism can comprise only one toggle lever leg as long as the toggle point is mounted in a guided manner in the direction of the longitudinal axis. Two toggle lever legs which are arranged symmetrically to the longitudinal axis however have the advantage that the transverse forces essentially cancel one another out and do not have to be accommodated by a guide.

The axial range of motion of the toggle point relative to a proximal bearing point of the spring element is preferably determined by the length of the guide opening in the spring element. The greater the length of the toggle lever legs, the shallower is the course of the curve of the force which is transmitted onto the transmission element in dependence on the position of the toggle point in the guide opening. Such a shallow force curve is desirable in order to achieve a constant as possible force which is transmitted onto the transmission element. Alternatively or additionally, a shallow force curve can be achieved by a low spring constant of the spring element, wherein however this cannot exceed as certain minimal amount for the desired biasing. It is likewise favourable for a shallow force curve if the minimal opening angle of the toggle lever mechanism is as large as possible, thus if the toggle point is at the proximal end of the guide opening, wherein here too the desired biasing sets an upper limit.

Optionally, the tool head can form a forceps, a pair of scissors and/or a pinching forceps.

Optionally, the second grip element can be rigidly connected or connectable to the shank.

Optionally, the first grip element can abut in a closure position when the first tool part has already abutted in a closure position without tissue which blocks. The tool head then cannot be overloaded without tissue which blocks. Moreover, by way of this, given small pieces of tissue which are to be gripped, pinched or cut, an effectively much shorter axial range of motion of the toggle point in the guide opening can result than its length would basically permit. Inasmuch as this is concerned, only a short portion of the force curve is of relevance for small pieces of tissue, such a portion beginning with the biasing force and then running as shallow as possible.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1a is a lateral view of an embodiment example of a surgical instrument which is disclosed herein, close to a closure position and

FIG. 1b is a lateral view of an embodiment example of a surgical instrument which is disclosed herein, in a greatly opened position (FIG. 1b);

FIG. 2 is a detailed lateral view of a proximal part of the surgical instrument which is shown in FIGS. 1a, b;

FIG. 3 is a detailed lateral view of the spring element of the surgical instrument which is shown in FIGS. 1a, b;

FIG. 4 is a detailed lateral view of the overload protection device of the surgical instrument which is shown in FIGS. 1a, b;

FIG. 5 is a representation of the force actions in the overload protection device of the surgical instruments which is shown in FIGS. 1a, b;

FIG. 6a is a representation of certain parameter values of the overload protection device of the surgical instrument which is shown in FIGS. 1a, b as well as a force-curve diagram; and

FIG. 6b is a representation of certain parameter values of the overload protection device of the surgical instrument which is shown in FIGS. 1a, b as well as a force-curve diagram.

### DESCRIPTION OF PREFERRED EMBODIMENTS

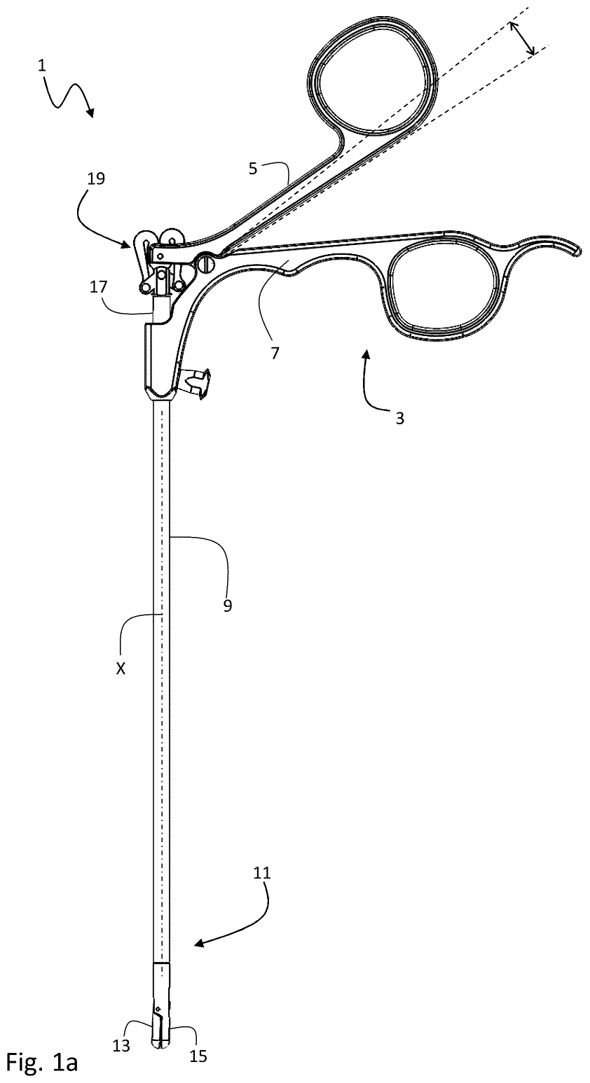

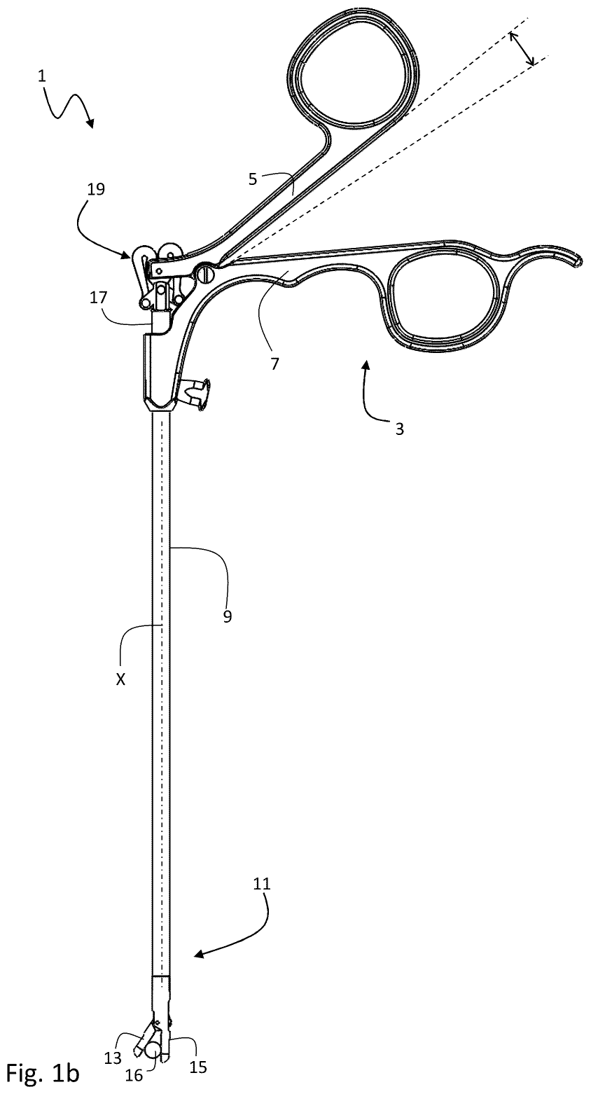

Referring to the drawings, a surgical instrument 1 in the form of a laparoscopic forceps is shown in FIGS. 1a,b, said forceps being almost closed in FIG. 1a and widely opened in FIG. 1b. The instrument comprises a manually grippable and actuatable actuation device 3 with two grip parts 5, 7 which can be gripped with one hand as with a pair of scissors. A first grip part 5 of the two grip parts 5, 7 can be moved relative to a second immovable grip part 7 of the two grip parts 5, 7 by the thumb of the user. For this, the first grip part 5 is pivotably mounted on the second grip part 7. A shank 9 extends distally from the immovable second grip part 7 along a longitudinal axis X of the surgical instrument 1 to a tool head 11. The tool head 11 in this embodiment example is a forceps head with two tool parts 13, 15 which here are configured as forceps jaw parts. At least a first 13 of the two tool parts 13, 15 is pivotably mounted with respect to the second 15 of the two tool parts 13, 15. As the case may be, both tool parts 13, 15 can be pivotably mounted relative to the shank 9. A relatively large piece of tissue 16 is shown between the two tool parts 13, 15 in FIG. 1b. The tissue piece 16 is too small to be seen in FIG. 1a.

A transmission element 17 which is axially displaceable in the shank 9 in a movable manner along a longitudinal axis X runs in the shank 9, said transmission element being in the form of a pull/push rod for transmitting a manual force from the first grip part 5 onto the first tool part 13 and/or second tool part 15. The transmission element 17 at the proximal side is coupled in a mounted manner to the first grip part 5 and at the distal side is coupled at least to the first tool part 13. A manually effected closure of the grip parts 5, 7 in the closure direction therefore effects a distal displacement of the transmission element 17 in the shank 9, said displacement in turn effecting a closing of the tool parts 13, 15. Accordingly, an opening of the grip parts 5, 7 in the opening direction effects a proximal displacement of the transmission element 17 in the shank 9 which in turn effects an opening of the tool parts 13, 15. The manual force of the user is therefore transmitted onto the tool head 11 in order to be able to move the tool parts 13, 15 in a targeted manner.

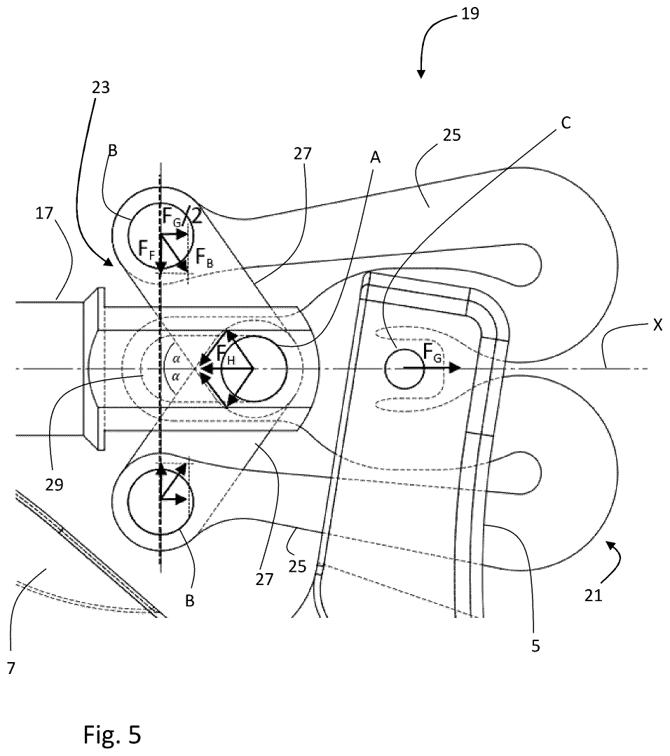

In order to protect the tool head 11 from an overloading with a too large manual force of the user, the surgical instrument 1 comprises an overload protection 19 with a spring element 21 for limiting the manual force which is transmitted onto the first tool part 13 in the closure direction. In contrast to overload protection devices which are known from the state of the art (SoA) concerning which a spring between the grip part and the transmission element is simply compressed starting from a certain force, the overload protection device 17 according to the present disclosure comprises a toggle lever mechanism 23, via which the spring element 21 biases the transmission element 17 in the closure direction, thus proximally towards the first grip part 5, with a biasing force F₀. For the biasing force F₀ in the closure direction, the following applies:

$${F}_{0} = {F}_{H} = 2 \frac{{F}_{F}}{tan {α}_{m i n}} ,$$

wherein F_{F} is a spring force which acts radially inwards at half the minimal opening angle αₘᵢₙ of the toggle lever mechanism 23 (see FIG. 5). Only when a proximal manual pulling force F_{G} which is exerted onto the spring element 21 via the first grip part 5 is larger than the biasing force F₀ does the opening angle 2a of the toggle lever mechanism 23 increase and the spring element 21 deflected.

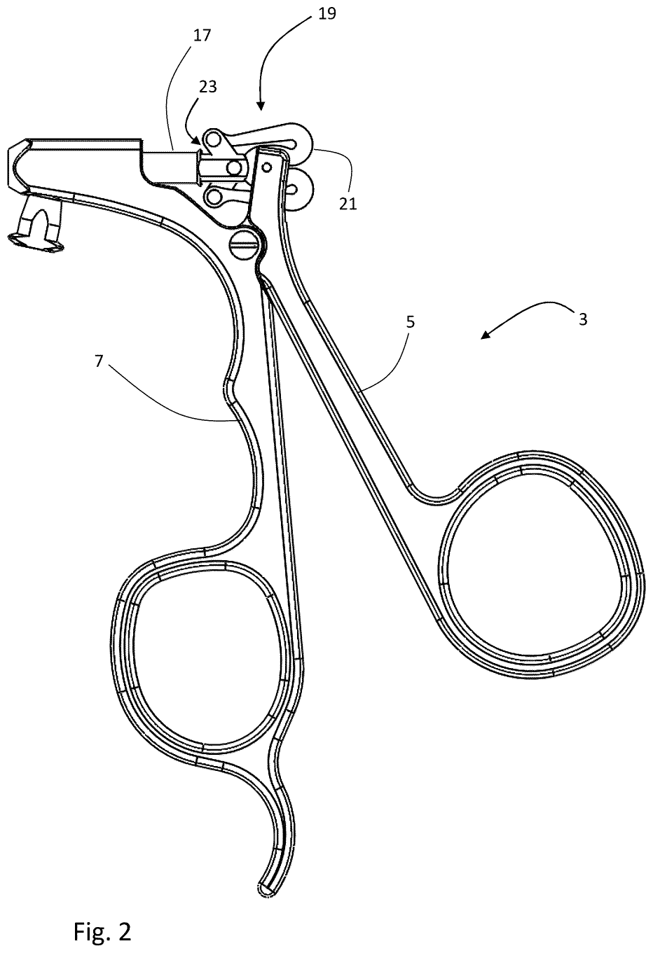

In FIG. 2, the proximal part of the surgical instrument 1 with the actuation device 3 and the overload protection device 19 is represented larger, in order to show the overload protection device 17 more clearly. If the first grip part 5 is pressed in the closure direction towards the second grip part 7, then a proximal bearing point C moves proximally. The spring element 21 is articulately connected to the first grip part 5 at the proximal bearing point C. By way of this, the spring element 21 is pulled proximally in the closure direction by the grip part 5. The spring element 21 is symmetrically curved in an M-shaped manner and comprises two spring bows 25 which extend distally from the proximal bearing point C laterally of the longitudinal axis at two opposite sides. A distal bearing point B is arranged each at the distal end of the spring bows 25.

The toggle lever mechanism 23 is arranged between the distal bearing points B and comprises two equally long toggle lever legs 27 which are articulately connected to one another in a toggle point A. The toggle point A as with the proximal bearing point C lies essentially on the longitudinal axis X, proximally of the distal bearing points B, so that the toggle lever mechanism 23 has a distally opened opening angle 2α. The toggle point A is a bearing point at the proximal end of the transmission element 17, so that the transmission element 17 only moves when the toggle point A moves.

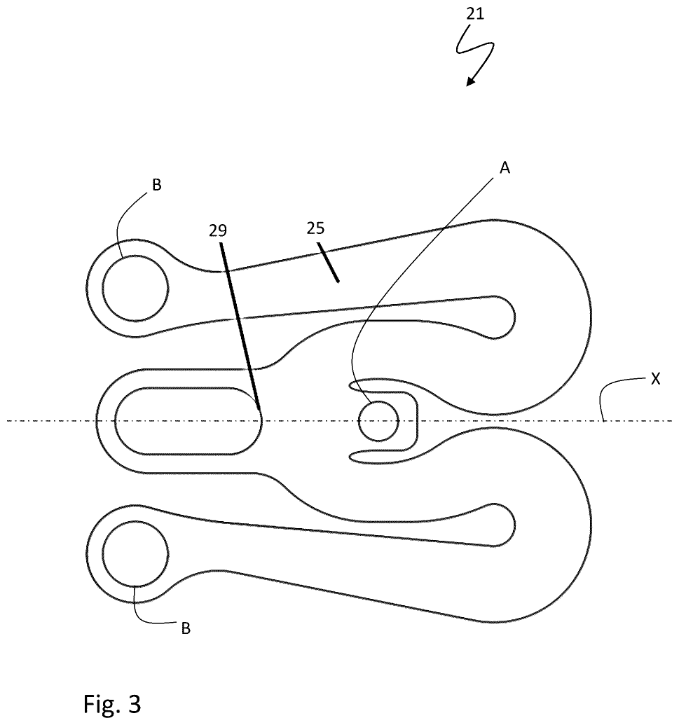

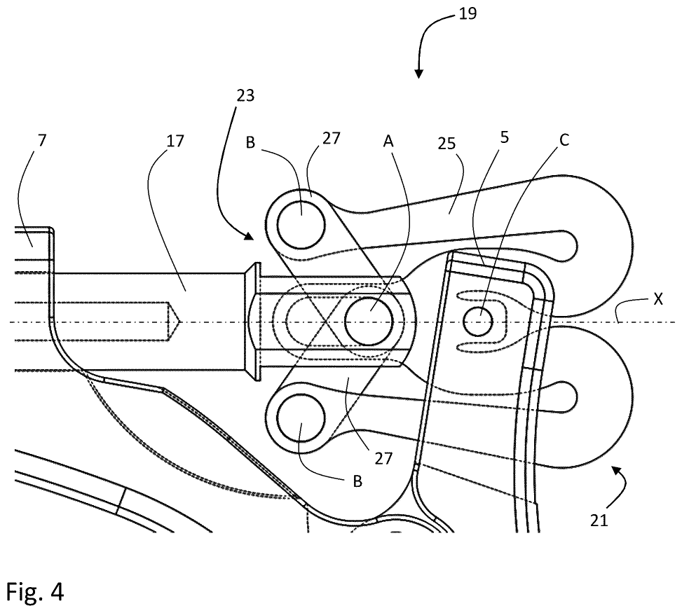

For the guided range of motion of the toggle point A relative to the spring element 21, the spring element 21 comprises a guide opening 29 in the form of an elongate hole which extends along the longitudinal axis X and in which the toggle point A is mounted. The elongate hole 29 can be better recognised in FIG. 3-6a. As long as no tissue 16 between the tool parts 13, 15 forms a resistance against closure, then the spring element 21 via the toggle lever mechanism 23 pulls the toggle point A proximally in the closure direction, without the toggle point A moving distally in the guide opening 29. The friction forces in the surgical instrument 1 for moving the tool parts 13, 15 should be so low that the overload protection device 19 is not activated as long as no tissue 16 between the tool parts 13, 15 forms a resistance against the closure.

The toggle point A in the proximal position is biased within the guide opening 29 by the spring element 21. This means that the length L of the toggle lever legs 27 and the position of the guide opening 29 is selected such that the spring bows 25 are already deflected somewhat and are subjected to a radially inwardly acting biasing force F₀ when the toggle point A is still situated within the guide opening 29 at the proximal end as is shown. In order to move distally within the guide opening 29, this biasing force F₀ must be overcome and the opening angle 2α of the toggle lever mechanism 23 widen against the spring force of the spring element 21. Herein, the distal bearing points B are pressed radially outwards against the spring force of the spring element 21.

It is important to understand that on the one hand the range of motion of the first grip part 5 is limited and on the other hand that the overload protection device 19 is not activated until tissue 16 between the tool parts 13, 15 forms a resistance against closure. Although there remains very little range of motion for the first grip part 5, a very small piece of tissue 16 between the tool parts 13, 15 can be held, pinched or cut with a desired minimal force due to the biasing force F0 in the overload protection device 19. For larger opening angles 2α of the toggle lever mechanisms 23, the manual force which is transmitted onto the transmission element 17 reduces. However, larger opening angles 2α of the toggle lever mechanism 23 only occur in the case of large, hard tissue pieces 16 which in practise play no significant role. Moreover, larger pieces of tissue 16 can be packed at least partly in the middle or proximal section of the tool parts 13, 15 where the gripping, pinching or cutting force is larger due to the shorter lever. The present disclosure is directed towards improving the tweezer grip of small pieces of tissue 16 at the outermost distal end of the tool parts 13, 15 without forgoing the overload protection, such a tweezer grip being commonly relevant in practise.

FIG. 5 illustrates the forces which prevail in the overload protection device 19. If a manual force F_{G} is exerted in the closure direction by the first grip part 5 in order to proximally pull the spring element 21 at the proximal bearing point C, then this force is divided onto both distal bearing points B, wherein half the force F_{G}/2 acts proximally in each case. The spring 20 force F_{F} of the spring element 21 acts radially inwards in each case, so that an intermediate force F_{B} upon the toggle point results along the toggle lever legs 27. As soon as a tissue piece 16 between the tool parts 13, 15 forms an adequate resistance counter to the closure, in order deflect the spring element 21, the force

$${F}_{H} = 2 {F}_{B} cos α = 2 {F}_{B} \frac{b}{L}$$

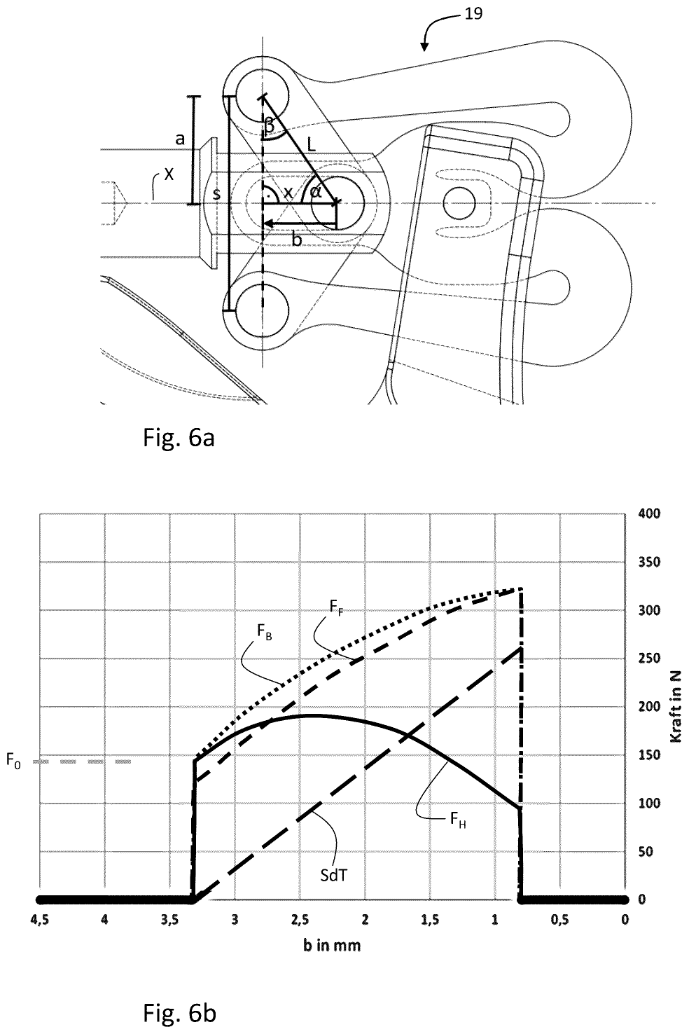

is transmitted onto the transmission element 17, wherein b is the momentary axial distance between the distal bearing points B and the toggle point A (see FIG. 6A).

A force-curve diagram of the spring force F_{F}, the intermediate force F_{B} and the force F_{H} which is transmitted onto the transmission element 17 in comparison to the transmitted force given a spring from the state of the art (SoA), as a function of the momentary axial distance b between the distal bearing points B and the toggle point A, is shown in FIG. 6b. Since the distance b reduces in the course of the activation of the overload protection device 19, b reduces to the right in the diagram. If the toggle point A is located at the proximal end of the guide opening 29, the distance b at the beginning of the activation of the overload protection device 19 is roughly 3.3 mm. The respective minimal opening angle 2α of the toggle lever mechanism 23 is at about 120°, i.e. half the opening angle a is minimally approx. 60°

If in the case of a large, hard tissue piece 16 between the tool parts 13, 15 the toggle point A abuts on the distal end of the guide opening 29 at maximal spring deflection a, then the distance b is roughly 0.8 mm. The guide opening 29 therefore permits the toggle point A a maximal path of 2.5 mm in the guide opening 29. The range of motion of the first grip part 5 can accordingly be restricted such that the proximal bearing point C is likewise only axially movable by 2.5 mm or less. Herewith, the spring element 21 whilst circumventing the toggle lever mechanism 23 at all events cannot introduce the proximal tensile force onto the transmission element 17 directly with the distal end of the guide opening 29. The first grip part 5 is therefore also abutting in the closure position at the distal end of the guide opening 29. In this situation, the spring deflection a as well as the opening angle 2α of the toggle lever mechanism 23 is simultaneously maximal at approx. 166°, i.e. half the opening angle α is maximally approx. 83°. The length L of the toggle lever legs in this example is about 6.6 mm.

FIG. 6b in a longitudinally dashed manner shows the course of the force of the transmitted force given a spring form the state of the art (SoA) as a straight line, which from the activation of the overload protection device increases continuously up to the maximal spring deflection. In contrast, a completely different course of the force F_{H} which is transmitted onto the transmission element 17 is achieved due to the biasing of the transmission element 17 in the closure direction by way of the biasing force F₀ of the spring element 21, said biasing being effected via the toggle lever mechanism 23. Although the spring force F_{F} and also the intermediate force

$${F}_{B} = \frac{{F}_{F}}{sin α}$$

drops with a decreasing distance b or increasing opening angle 2α of the toggle lever mechanism 23, the force F_{H} which is transmitted onto the transmission element 17 however only increases in a shallow manner and achieves a maximum at a distance b of approx. 2.4 mm and drops for smaller distances b. This is due to the fact that the cosine term in F_{H}=2F_{B} cos a greatly drops in the angular range of 60° to 83°. The length L of the toggle lever legs 25, the spring constant of the spring element 21 as well as the minimal opening angle 2α of the toggle lever mechanism 23, here αₘᵢₙ=60° are selected here such that the force F_{H} which is transmitted onto the transmission element 17 even drops being the level of the biasing force F₀ below a distance b of approx. 1.2 mm. It is to be noted that in the force equilibrium F_{G}=F_{H}=2F_{B} cos α is always the case, thus

$${F}_{B} = \frac{{F}_{F}}{sin α} = \frac{{F}_{G}}{2 cos α} .$$

Since the curve regions with a small distance b or large opening angles 2α of the toggle lever mechanism 23 only occur in the case of large, hard pieces of tissue 16 between the tool parts 13, 15, the initial curve region with large distances or small opening angles 2α of the toggle lever mechanism 23 is of particular relevance in practise. Given small tissue pieces 16, specifically the overload protection device 19 is not activated in the closure direction over a large part of the path which is available to the first grip part 5. If for example in the extreme case the tissue piece 16 is so small that so little space remains for the grip part 5 until abutting on the second grip part 7 that it can pull the proximal bearing point C proximally by only 1 mm, then the force curve is only of relevance only in the range of the distance b between 3.3 mm and 3.2 mm. Given a simple spring compression of the state of the art (SoA) as an overload protection, hardly any manual force F_{H} at all could be transmitted in the initial region (see longitudinally dashed curve).

In order to keep the curve of the force F_{H} which is transmitted onto the transmission element 17 as shallow as possible, the length L of the toggle lever legs 25 is to be selected as largely as possible within the scope of other design boundaries. The biasing force F₀ should be as high as possible given a low as possible spring constant, in order to permit a forceful as possible tweezer grip of small tissue pieces 16. A high minimal opening angle 2α of the toggle lever mechanisms 23 is advantageous in order to keep the maximal spring force Fr as low as possible so as to keep the structural loading of the system components as low as possible.

While specific embodiments of the invention have been shown and described in detail to illustrate the application of the principles of the invention, it will be understood that the invention may be embodied otherwise without departing from such principle

### LIST OF REFERENCE NUMERALS

1 surgical instrument
3 actuation device
5 first grip part
7 second grip part
9 shank
11 tool head
13 first tool part
15 second tool part
16 tissue piece
17 transmission element
19 overload protection device
21 spring element
23 toggle lever mechanism
25 spring bow
27 toggle lever legs
29 guide opening
A toggle point
B distal bearing point
C proximal bearing point
X longitudinal axis
L length of the toggle lever legs
B axial distance between the toggle point and the distal bearing point
F_{H} manual force transmitted onto the transmission element
F_{G} manual force acting upon the proximal bearing point
F_{F} spring force
F_{B} intermediate force
F₀ biasing force
2α opening angle of the toggle lever mechanism

1. A surgical instrument for the surgical gripping, pinching and/or cutting of tissue within an organic body, the surgical instrument comprising:
an actuation device comprising two grip parts, wherein at least a first of the two grip parts is movably mounted relative to a second of the two grip parts in a translatory and/or rotatory manner in an opening and closure direction,
a tool head comprising two tool parts, wherein at least a first of the two tool parts is movably mounted relative to a second of the two tool parts in a translatory and/or rotatory manner in an opening and closure direction,
a shank which extends along a longitudinal axis between the actuation device and the tool head, wherein a proximal end of the shank is connected or connectable to the actuation device, and wherein a distal end of the shank is connected or connectable to the tool head, wherein the shank comprises a transmission element which is axially movable in an opening and closure direction for transmitting a manual force from the first grip part onto the first tool part, and
an overload protection device comprising a spring element for limiting the manual force which is transmitted onto the first tool part in the closure direction, wherein the overload protection device comprises a toggle lever mechanism, via which the spring element biases the transmission element in the closure direction.

2. A surgical instrument according to claim 1, wherein the toggle lever mechanism is articulately connected to the transmission element in a toggle point.

3. A surgical instrument according to claim 1, wherein the toggle lever mechanism comprises two distal bearing points which are radially distanced to the longitudinal axis and via which the toggle lever mechanism is articulately connected in each case to one of two spring bows of the spring element.

4. A surgical instrument according to claims 2, wherein the toggle point is arranged proximally of the distal bearing points.

5. A surgical instrument according to claim 1, wherein the spring element comprises a proximal bearing point, via which the spring element is articulately connected to the first grip part.

6. A surgical instrument according to claim 5, wherein the toggle lever mechanism is articulately connected to the transmission element in a toggle point, and wherein the proximal bearing point is arranged proximally of the toggle point.

7. A surgical instrument according to claim 1, wherein the spring element is coupled to the first grip part in a manner such that, given a movement of the first grip part in the closure direction, the spring element spreads radially outwards counter to an intrinsic spring tension amid an opening of the toggle lever mechanism as soon as the further movement of the first tool part is blocked in the closure direction by tissue.

8. A surgical instrument according to claim 1, wherein the spring element comprises two spring bows which on opposite sides extend laterally of the longitudinal axis from the first grip part to the toggle lever mechanism.

9. A surgical instrument according to claim 2, wherein the spring element comprises a guide opening which extends along the longitudinal axis and in which the toggle point is mounted in an axially movably guided manner.

10. A surgical instrument according to claim 1, wherein the spring element is biased with a biasing force when the toggle lever mechanism is at a minimal opening angle, wherein the opening angle of the toggle lever mechanism widens counter to the biasing force as soon as the further movement of the first tool part is blocked in the closure direction by way of tissue.

11. A surgical instrument according to claim 10, wherein the manual force which is transmitted onto the first tool part in the closure direction is greater than the biasing force in a first opening angle region of the toggle lever mechanism and is lower than the biasing force in a second opening angle region of the toggle lever mechanism which lies above the first opening angle region.

12. A surgical instrument according to claim 2, wherein the toggle lever mechanism comprises two equally long toggle lever legs which are articulately connected to one another in the toggle point, wherein the length of the toggle lever legs is larger than an axial range of motion of the toggle point relative to a proximal bearing point of the spring element, via which bearing point the spring element is articulately connected to the first grip element.

13. A surgical instrument according to claim 1, wherein the tool head forms a forceps, a pair of scissors and/or a pinching forceps.

14. A surgical instrument according to claim 1, wherein the second grip element is rigidly connected or connectable to the shank.

15. A surgical instrument according to claim 1, wherein the first grip element abuts in a closure position when the first tool part has already abutted in a closure position without tissue which blocks.
